(19) United States
(12) Patent Application Publication
(10) Pub. No.: US 2018/0070065 A1
(43) Pub. Date: Mar. 8, 2018

(54) IMAGE PROCESSING METHOD AND IMAGE PROCESSING DEVICE

(71) Applicants: INVENTEC (PUDONG) TECHNOLOGY CORPORATION, Shanghai City (CN); INVENTEC CORPORATION, Taipei City (TW)

(72) Inventor: Feng-Shan CHEN, Taipei City (TW)

(73) Assignees: INVENTEC (PUDONG) TECHNOLOGY CORPORATION, Shanghai City (CN); INVENTEC CORPORATION, Taipei City (TW)

(21) Appl. No.: 15/346,878

(22) Filed: Nov. 9, 2016

(30) Foreign Application Priority Data
Sep. 5, 2016 (CN) 201610801101.8

(51) Int. Cl. H04N 9/64; H04N 1/60; G06K 9/62; G06T 11/60; G09B 21/00; A61B 3/06

(52) U.S. Cl. CPC H04N 9/64; H04N 1/6008; G06K 9/6202; G06T 2200/24; G09B 21/008; A61B 3/066; G06T 11/60

(57) ABSTRACT

An image processing method and an image processing device are disclosed. The image processing method includes following steps: acquiring a first image, wherein the first image includes multiple pixels; acquiring color compensating information from a user interface; calibrating the value of each of the multiple pixels based on the color compensating information to generate a second image.

### CROSS-REFERENCE TO RELATED APPLICATIONS

This non-provisional application claims priority under 35 U.S.C. §119(a) on Patent Application No(s). 201610801101.8 filed in China on Sep. 5, 2016, the entire contents of which are hereby incorporated by reference.

### BACKGROUND

### Technical Field

This disclosure relates to an image processing method and an image processing device, and more particularly to an image processing method for color compensating and an image processing device with the function of color compensating.

### Related Art

Generally, the patients with mild blindness have the decreased ability to cognize some kinds of color. For example, for protanomaly patients or deuteranomaly patients, it's hard to cognize red or green things in the reality or in the figures is hard for the. This trouble causes the patients much convenience. While cooking, the patients might have trouble determining whether the food is undercooked; while picking clothing, the patients might have difficulty in color matching; and while buying fruit and vegetable, the patients often buy the wrong thing. For example, the patients cannot distinguish green peppers from red peppers.

The conventional assistant devices for color blindness are usually the glasses or the contact lenses based on the theory of polarization or color compensation to assist the patient with color blindness in discriminate colors. However, these devices just can aim at a single color (e.g., red or green) to compensate. Therefore, the patients with vision deficiency in several colors (e.g., protanomaly and deuteranomaly which are the most common vision deficiency) just can choose a single color to compensate. Furthermore, via the above kind of assistant devices for color blindness, the patients might see the scene without the original color.

### SUMMARY

This disclosure provides an image processing method, including the following steps: acquiring a first image which comprises multiple pixels; acquiring color compensating information from a user interface; and calibrating a pixel value of each of the multiple pixels based on the color compensating information to generate a second image.

This disclosure provides an image processing device, including an image acquiring module and an operating module. The operating module is coupled to the image acquiring module. The image acquiring module is used for acquiring a first image which has multiple pixels. The operating module is used for acquiring color compensating information from a user interface, and calibrating the value of each pixel of the first image based on the color compensating information to generate a second image.

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. 1 is a schematic diagram of an image processing device in an embodiment;

FIG. 2A and 2B are schematic diagrams of a user interface with function of color compensating in an embodiment;

FIG. 3A and 3B are schematic diagrams of color test plates in an embodiment;

FIG. 4A and 4B are schematic diagrams of a user interface with function of color marking in an embodiment;

FIG. 5 is a schematic diagram of a user interface with function of color differentiation in an embodiment; and

FIG. 6 is a flow chart of an image processing method in an embodiment.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawings.

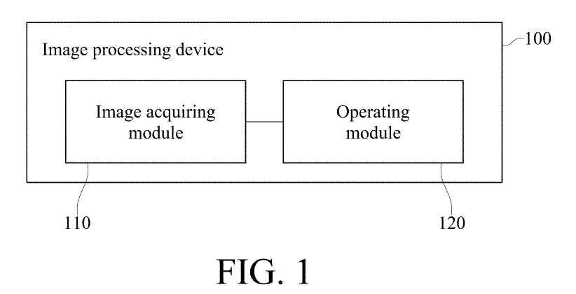

FIG. 1 is a schematic diagram of an image processing device in an embodiment. As show in FIG. 1, an image processing device 100 includes an image acquiring module 110 and an operating module 120. The operating module 120 is coupled to the image acquiring module 110. The operating module 120 and the image acquiring module 110 can be implemented by chips with various kinds of functions or microprocessors. This disclosure does not intend to limit how to implement the modules.

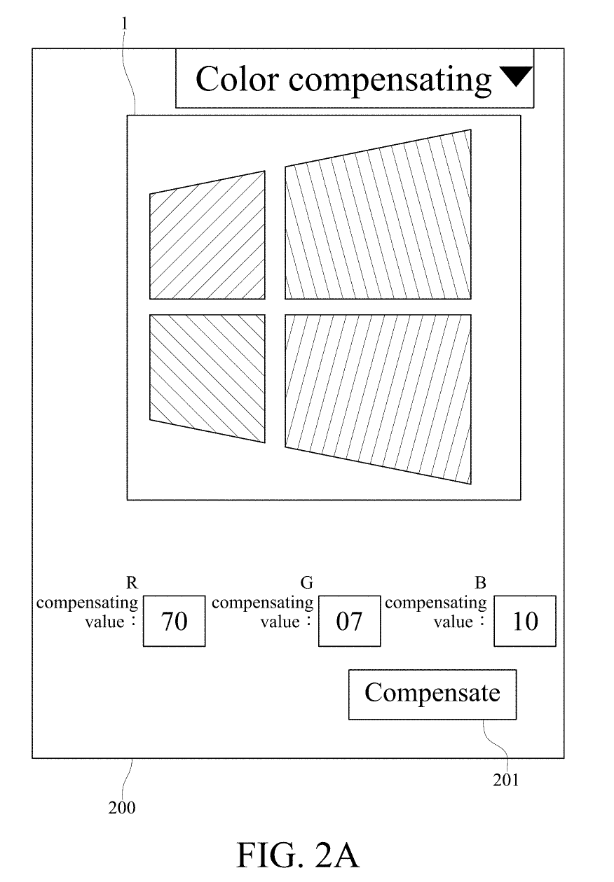

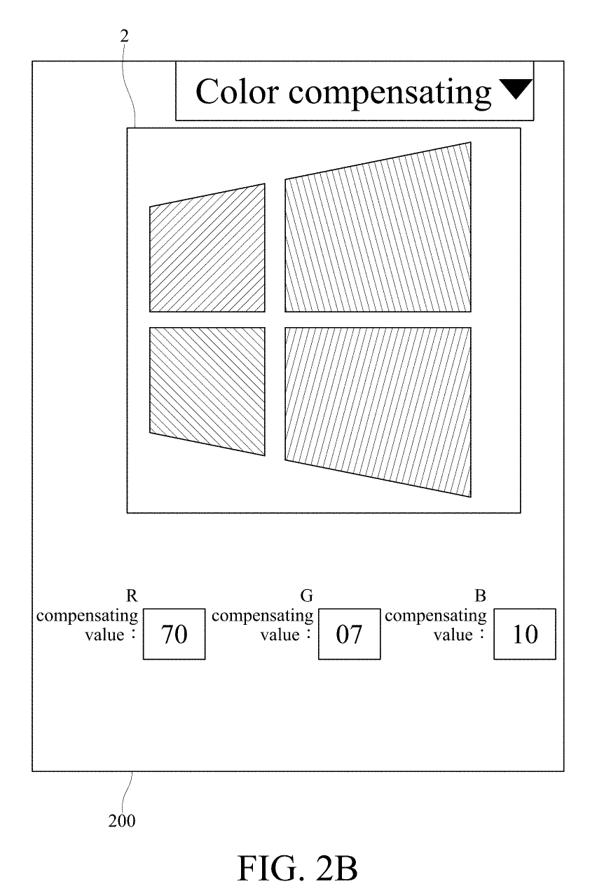

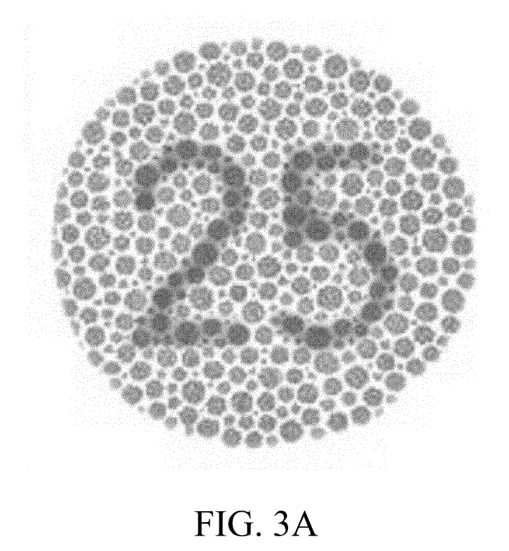

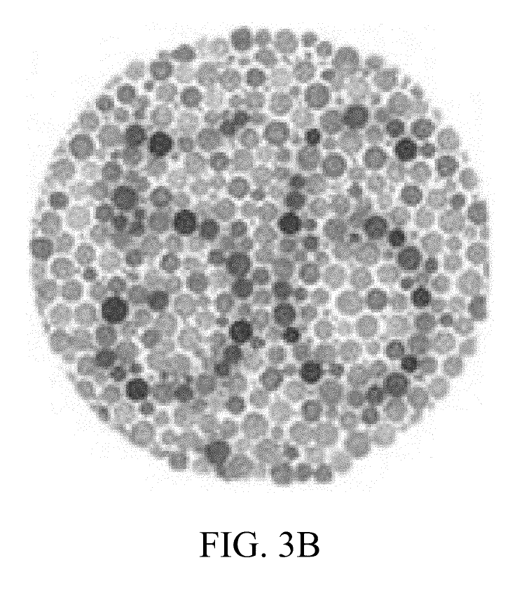

FIG. 2A and 2B are schematic diagrams of a user interface with function of color compensating in an embodiment. Please refer to FIG. 1, FIG. 2A and FIG. 2B.

The image acquiring module 110 is used for acquitting a first image 1 with multiple pixels. In an embodiment, an image stream is acquired by a camera of a handheld electronic device or a wearable device. Then, the color of the image stream is converted to generate the first image 1. For example, the process of color conversion mentioned above is converting the image stream from its original color space to the RGB color space, which is composed of red, green and blue. As shown in FIG. 2A, the first image 1 is displayed via a user interface 200. In another embodiment, the process of color conversion is converting the image stream from its original color space to the CMYK color space, the CIE 1931 color space or other color space to generate the first image 1. This disclosure does not intend to limit the process of color conversion.

The operating module 120 is used for acquiring color compensating information from a user interface 200 and calibrating the value of each pixel of the first image 1 based on the color compensating information to generate a second image 2. For example, as shown in FIG. 2B, the second image 2 is displayed via the user interface 200. In another embodiment, the second image 2 is outputted via a wearable electronic device, such as a virtual reality device, or other electronic device to a user. For RGB color space, the color compensating information includes R compensating value, G compensating value and B compensating value, which are defined as the compensation value of red, green and blue respectively. As shown in FIG. 2A, the R compensating value is set as 70, the G compensating value is set as 07, and the B compensating value is set as 10. The color compensation is executed as pressing a button 201.

As another example, for protanomaly patients or deuteranomaly patients, their ability to cognize red and green is weaker, so the R compensating value can be set as +30 and the G compensating value can be set as +20. As the RGB value of one of the pixels in the first image 1 is [20, 100, 200], the RGB value relative to the pixel mentioned above in the second image 2 after calibrating is [50, 120, 200]. As a result, the feelings of red and green of the protanomaly patients or the deuteranomaly patients can be enhanced at the same time. The following describes a method for getting color compensating information.

In an embodiment, the operating module 120 in advance outputs multiple color test plates, as shown in 3A and 3B. Then, the operating module 120 acquires a test result relative to each color test plate from the user interface and generates the color compensating information according to the test result. The color test plate mentioned above is a test plate of partial color blindness or total color blindness. In addition, the operating module 120 selectively outputs one of the various color plates for detecting different levels of color blindness in each test. The operating module 120 gets the relative test result successively to generate the most fitting R compensating value, G compensating value, or B compensating value.

For example, when the operating module 120 detects that a user probably has protanomaly, who has trouble cognizing red, the operating module 120 outputs a color test plate which represents more serious protanomaly (e.g., the red in the color test plate is brighter). When the user answers correctly to the color test plates representing more serious protanomaly, the operating module 120 outputs a color test plate which represents milder protanomaly (e.g., the red in the color test plate is not as bright as the red in the previous color test plate). When the user answers wrong to the color test plates representing milder protanomaly, the real severity of protanomaly of the user is speculated between the two indices relative to the above two color test plates. As a result, the real feeling of the R value of the user can be approached successively to get the most accurate R compensating value of the color compensating information. The process of getting the G compensating value or the B compensating value is similar to the above description, so the related details are not described again.

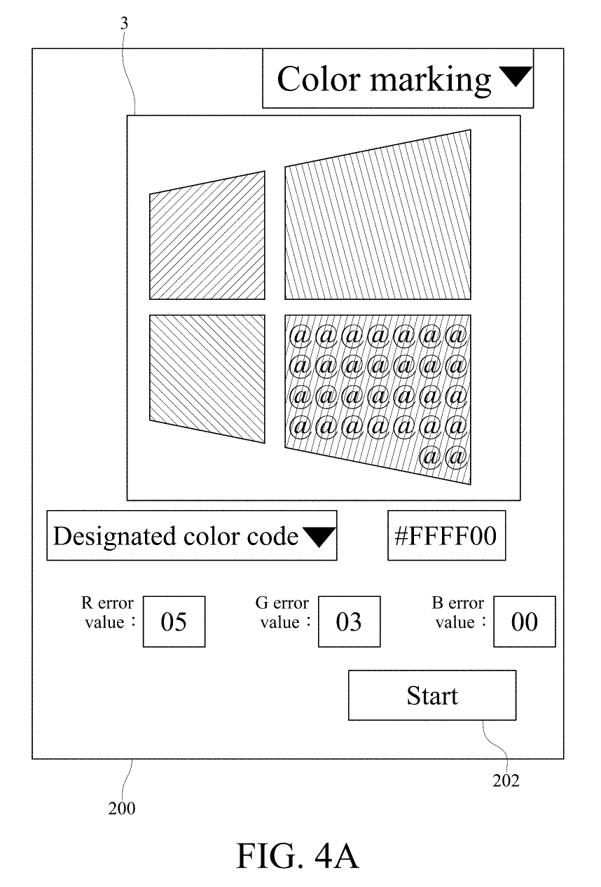

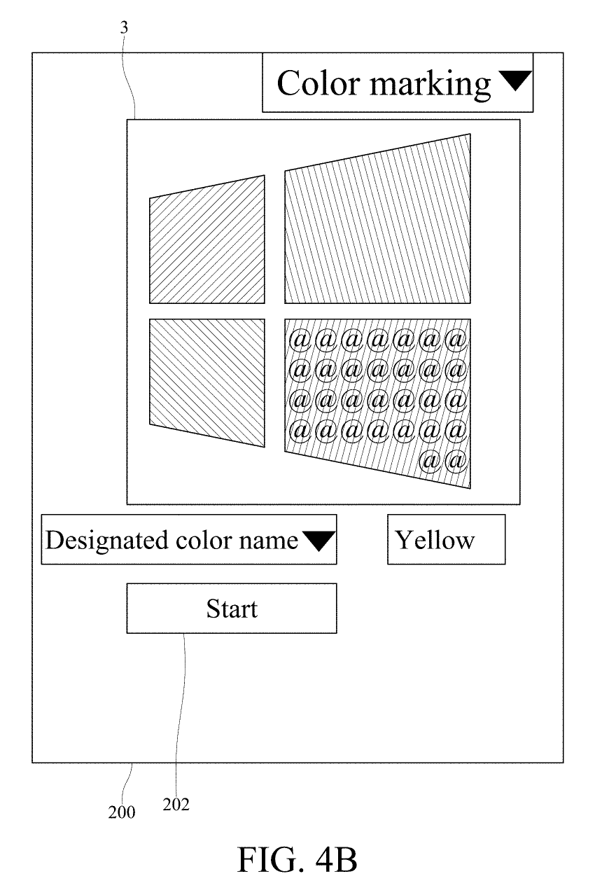

FIG. 4A and 4B are schematic diagrams of a user interface with function of color marking in an embodiment. Please refer to FIG. 1, FIG. 4A, and FIG. 4B.

In an embodiment, the operating module 120 acquires designated color information from the user interface 200, and determines whether at least one of the multiple pixels of the first image 1 is matched to the designated color information. When at least one of the multiple pixels of the first image 1 is matched to the designated color information, the operating module 120 acquires a first part relative to the pixel matched to the designated color information, generates a hint message relative to the first part, and combines the first image 1 with the hint message to generate a third image 3. The designated color information mentioned above includes a designated color code, a designated color name. In an embodiment, the designated color information also includes an error tolerance of color.

For example, the user can set the designated color code as #FFFF00 to search if there is any relative first part with the designated color code #FFFF00 in the first image 1 via the operating module 120. Furthermore, as setting the error tolerance of color, the user can set the error value of red as ±5, the error value of green as ±3, and the error value of blue as 0. Therefore, when the user presses the button 202 and there is the relative first part with the range of RGB value, [250·255, 117˜123, 200], in the first image 1, the operating module 120 generates a relative hint message and combine the first image 1 with the hint message to generate the third image 3, as shown in 4A. In an embodiment, the hint message is a pattern for making the first part more clear to assist the user in finding the first part. For example, the pattern “@” shown in FIG. 4A is a kind of hint. This disclosure does not intend to limit the form or the arrangement of the pattern mentioned above.

In an embodiment, as shown in FIG. 4B, the user sets that the designated color name includes the word “yellow” to search that if there is any relative first part in the first image via the operating module 120. The color corresponding to the color name “yellow” is, for example, mustard (color code: #CCCC4D, RGB value: [204, 204, 77]), moon yellow (color code: #FFFF4D, RGB value: [255, 255, 77]), olive (color code: #808000, RGB value: [128, 128, 0]), and canary yellow (color code: #FFFF00, RGB value: [255, 255, 0]). Therefore, when the user presses the button 202 and there is the relative first part with the designated color name including the word “yellow”, the operating module 120 generates a relative hint message, such as the pattern making the first part more clear (e.g., pattern “@”), and combines the first image 1 with the hint message to generate the third image 3.

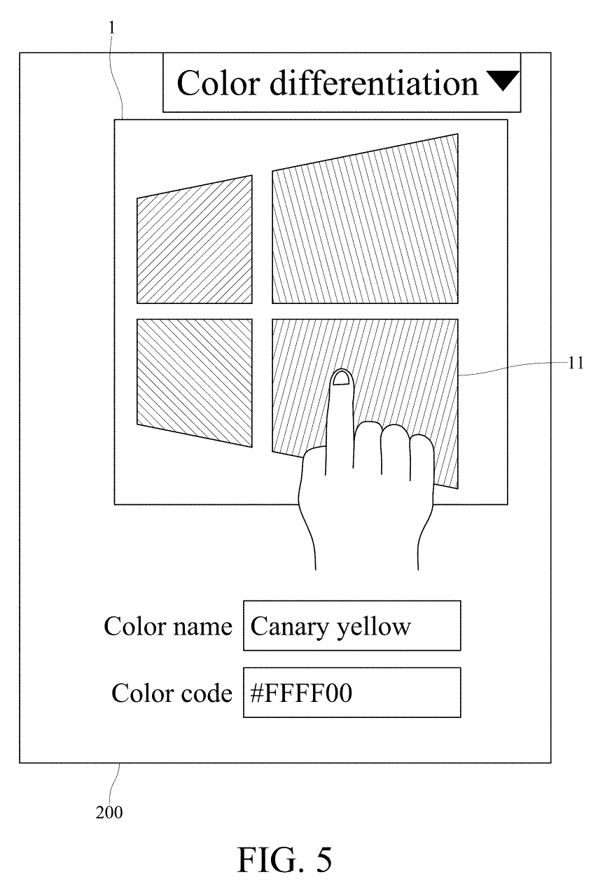

FIG. 5 is a schematic diagram of a user interface with function of color differentiation in an embodiment. Please refer to FIG. 1 and FIG. 5.

In an embodiment, the operating module 120 acquires a second part of the first image 1 from the user interface 200 and generates color information relative to at least one of pixels of the second part wherein the color information includes a color code or a color name.

For example, as shown in FIG. 5, when a user cannot cognize which color the element 11 in the first image 1 is, the user selects the element 11 via the user interface 200. Then, the operating module 120 generates a color code or a color name relative to the element 11, which is the second part.

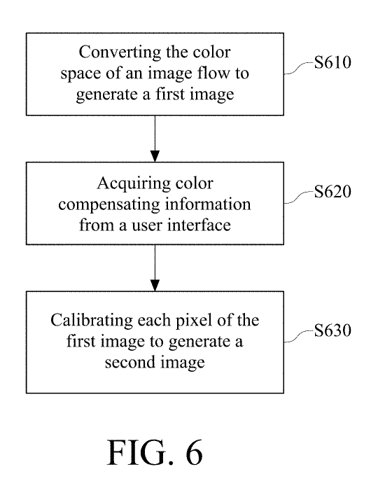

FIG. 6 is a flow chart of an image processing method in an embodiment. As shown in FIG. 6, the image processing method in this embodiment includes steps S610˜S630.

In step S610, the image stream has a color conversion to acquire a first image.

In step S620, color compensating information is acquired from a user interface.

In step S630, the second image is generated by calibrating the value of each pixel of the first image based on the color compensating information. The details of these steps are described before and are not explained again.

In view of the above description, in an embodiment of the disclosure, color compensating information can be generated by acquiring the test result relative to each color test plate one by one. Therefore, the real feeling of RGB value can be approached successively to get the most accurate compensating value. Then, the value of each pixel of a first image is calibrated based on the compensating value to generate a second image. In another embodiment, a third image is generated by acquiring the first part, which is matched to designated color information and an error tolerance of color and the relative hint message, to make the first part more clear. In yet another embodiment, by designating a second part of the first image and generating a color code or a color name which is relative to the second part, the users are assisted in feeling and cognizing a designated color.

While this disclosure is described in terms of several embodiments above, these embodiments do not intend to limit this disclosure. It is therefore intended that the following appended claims be interpreted as including all such alterations, permutations, and equivalents as fall within the true spirit and scope of the present disclosure.

What is claimed is:

1. An image processing method, comprising:
acquiring a first image comprising a plurality of pixels;
acquiring color compensating information from an user interface; and
calibrating a pixel value of each of the plurality of pixels based on the color compensating information to generate a second image.

2. A image processing method according to claim 1, further comprising:
outputting a plurality of color test plates;
acquiring a test result relative to each of the plurality of color test plates from the user interface; and
generating the color compensating information according to the test result.

3. The image processing method according to claim 1, further comprising:
acquiring designated color information from the user interface;
determining whether at least one of the plurality of pixels of the first image is matched to the designated color information;
when at least one of the plurality of pixels of the first image is matched to the designated color information, acquiring a first part relative to the pixel matched to the designated color information, and generating a hint message relative to the first part; and
combining the first image with the hint message to generate a third image;
wherein the designated color information comprises a designated color code or a designated color name.

4. The image processing method according to claim 3, wherein the designated color information comprises an error tolerance of color.

5. The image processing method according to claim 1, further comprising:
acquiring a second part of the first image from the user interface; and
generating color information relative to at least one of pixel of the second part;
wherein the color information comprises a color code or a color name.

6. An image processing device, comprising:
an image acquiring module used for acquiring a first image, wherein the first image comprises a plurality of pixels; and
an operating module coupled to the image acquiring module, used for acquiring color compensating information from an user interface and calibrating a pixel value of each of the plurality pixels of the first image based on the color compensating information to generate a second image.

7. The image processing device according to claim 6, wherein the operating module further outputs a plurality of color test plates, acquires a test result relative to each of the plurality of color test plates from the user interface, and generates the color compensating information based on the test result.

8. The image processing device according to claim 6, wherein the operating module further acquires designated color information from the user interface, determines whether at least one of the plurality of pixels of the first image is matched to the designated color information, and when at least one of the plurality of pixels of the first image the first image is matched to the designated color information, acquires a first part relative to the pixel matched to the designated color information and generates a hint message relative to the first part, and combines the first image with the hint message to generate a third image, wherein the designated color information comprises a designated color code or a designated color name.

9. The image processing device according to claim 8, wherein the designated color information comprises an error tolerance of color.

10. The image processing device according to claim 6, wherein the operating module further acquires a second part of the first image from the user interface and generates color information relative to at least one of pixel of the second part, wherein the color information comprises a color code or a color name.
